(19) United States
(12) Patent Application Publication
(10) Pub. No.: US 2007/0111327 A1
(43) Pub. Date: May 17, 2007

(54) Methods of detecting lubricin

(76) Inventor: Gregory D. Jay, Norfolk, MA (US)

Correspondence Address: CLARK & ELBING LLP, 101 FEDERAL STREET, BOSTON, MA, 02110, US

(21) Appl. No.: 11/429,591

(22) Filed: May 5, 2006

Related U.S. Application Data
(60) Provisional application No. 60/678,025, filed on May 5, 2005.

(51) Int. Cl. G01N 33/543

(52) U.S. Cl. 436/518

(57) ABSTRACT

The invention features methods of detecting lubricin in a sample, such as, for example, the synovial fluid of a patient. The invention also features methods of diagnosing or detecting a degenerative joint condition or measuring a patient's response to a treatment for a degenerative joint condition.

### CROSS-REFERENCE TO RELATED APPLICATIONS

This application claims the benefit of the filing date of U.S. Provisional Application No. 60/678,025, filed on May 5, 2005, which is incorporated herein by reference.

### BACKGROUND OF THE INVENTION

The present invention relates to the detection of lubricin in synovial fluid and methods of diagnosing injured connecting tissue.

Lubricin, also known as proteoglycan 4 (PRG4), articular cartilage superficial zone protein (SZP), megakaryocyte stimulating factor precursor, or tribonectin (Ikegawa et al., Cytogenet. Cell. Genet. 90:291-297, 2000; Schumacher et al., Arch. Biochem. Biophys. 311:144-152, 1994; Jay et al., J. Rheumatol. 27:594-600, 2000; and Jay, WIPO Int. Pub. No. WO 00/64930) is a mucinous glycoprotein found in the synovial fluid (Swann et al., J. Biol. Chem. 256:5921-5925, 1981). Lubricin provides boundary lubrication of congruent articular surfaces under conditions of high contact pressure and near zero sliding speed (Jay et al., J. Orthop. Res. 19:677-87, 2001). These lubricating properties have also been demonstrated in vitro (Jay, Connect. Tissue Res. 28:71-88, 1992). Cells capable of synthesizing lubricin have been found in synovial tissue and within the superficial zone of articular cartilage within diarthrodial joints (Jay and Cha, J. Rheumatol., 26:2454-2457, 1999).

In U.S. patent application Ser. No. 10/038,694 are described methods of promoting lubrication between two juxtaposed biological surfaces using lubricin, or fragments thereof. In PCT Publication No. WO 00/64930 are described lubricin (tribonectin) analogs and methods for lubricating a mammalian joint. In a recent report (Englert et al., Trans. Orthop. Res. 29:189, 2003), the reduction of integration of opposing cartilage surfaces by components in synovial fluid was described and it was suggested that this reduction in integration was, at least in part, lubricin (SZP) mediated. In U.S. Pat. No. 6,720,156 is described a monoclonal antibody to lubricin (SZP), methods for detecting lubricin, and methods for diagnosing degenerative conditions using an antibody specific for lubricin.

Given the importance of lubricin to the lubrication of articulating mammalian joints, what is needed are improved methods for the detection and/or quantitation of lubricin in synovial fluid in order to assess the risk or extent of joint damage.

### SUMMARY OF THE INVENTION

Accordingly, in a first aspect, the present invention features a method for detecting lubricin in a sample that includes the steps of (a) immobilizing on a solid surface a first substance that binds to a peptide sequence of lubricin, (b) contacting the solid surface that has the first substance immobilized thereto with a sample suspected of containing lubricin under conditions in which lubricin binds to the first substance, (c) contacting the solid surface with a second substance that binds to a carbohydrate moiety of lubricin under conditions in which the carbohydrate binds to the second substance, and (d) detecting lubricin in the sample by observing the binding of the second substance to the solid surface.

In a second aspect, the invention features a method for detecting lubricin in a sample that includes the steps of (a) immobilizing on a solid surface a first substance that binds to a carbohydrate moiety of lubricin, (b) contacting the solid surface that has the first substance immobilized thereto with a sample suspected of containing lubricin under conditions in which lubricin binds to the first substance, (c) contacting the solid surface with a second substance that binds to a peptide sequence of lubricin under conditions in which the peptide sequence binds to the second substance, and (d) detecting lubricin in the sample by observing the binding of the second substance to the solid surface.

For any of the methods of the invention, non-limiting examples of samples that may contain lubricin include synovial fluid, synovium, tendon, tendon sheath, ligament, meniscus, intervertebral disc, chondrocytes, or articular cartilage. Desirably, the sample is synovial fluid

Non-limiting examples of substances that bind to a peptide sequence of lubricin include those substances that bind to a lubricin sequence selected from the group consisting of SEQ ID NO. 2, SEQ ID NO. 3, SEQ ID NO. 4, SEQ ID NO. 5, SEQ ID NO. 6, SEQ ID NO. 7, SEQ ID NO. 8, and SEQ ID NO. 9. Desirably, the lubricin sequence that is bound is SEQ ID NO. 2. Non-limiting examples of substances that bind to a lubricin carbohydrate include those substances that bind to a mucin carbohydrate, such as, for example, peanut agglutinin, or those that bind to chondroitin. Other non-limiting examples of substances that bind to a lubricin peptide or a lubricin carbohydrate include monoclonal or polyclonal antibodies, or nucleic acids, such as, for example, an RNA-based aptamer developed for such a purpose.

A salient feature of an assay of the invention is that it is a sandwich-based assay in which one assay component binds to a lubricin peptide sequence and one assay component binds to a lubricin carbohydrate moiety. Inclusion of both of these components ensures that assay detects non-degraded lubricin. This is important, as it has been shown that the abundance of negatively charged sugars in the lubricin carbohydrate domain contributes to lubricin's boundary lubricating abilities due to strong repulsive hydration forces (Jay, Connect. Tissue Res. 28:71-88, 1992). Non-limiting examples of sandwich-based assays that are useful in the methods of the invention include, without limitation, sandwich ELISAs, sandwich Western blotting assays, and sandwich immunomagnetic detection assays. These assays generally include reporter groups with detectable labels, such as, for example, radionuclides (e.g., ¹²⁵I, ¹³¹I, ³⁵S, ³H, ³²P, ³³P, or ¹⁴C); fluorescent moieties (e.g., fluorescein, rhodamine, or phycoerythrin); luminescent moieties (e.g., nanoparticles), or enzymes (e.g., alkaline phosphatase or horseradish peroxidase). The products of reactions catalyzed by appropriate enzymes can be, without limitation, fluorescent, luminescent, or radioactive, or they may absorb visible or ultraviolet light. A preferred reporter group of the invention includes horseradish peroxidase.

Also included in the methods of the invention are those sandwich assays in which a reporter group need not be included. These assays can distinguish between a complex in which the first sandwich component of the assay (the capture substance) has reacted with the ligand (i.e., lubricin) and a complex in which both sandwich components of the assay (the capture and detection substances) have reacted with the ligand. In one non-limiting example, the difference between these two complexes can be determined by a surface plasmon resonance measurement. As before, the capture substance can be either a substance that binds to a lubricin peptide sequence or to a lubricin carbohydrate moiety, with the detection substance being the complementary lubricin carbohydrate-binding substance or lubricin peptide-binding substance, respectively.

In another aspect, the invention features a method for detecting or diagnosing a degenerative connecting tissue condition in a subject that includes the steps of (a) obtaining a connecting tissue sample from the subject, (b) detecting lubricin in the tissue sample and in a control sample by the method of either the first or second aspect of the present invention, (c) comparing the amount of lubricin in the tissue sample with the amount of lubricin in the control sample, and (d) determining that the subject has a degenerative connecting tissue condition if the amount of lubricin in the tissue sample is modulated compared to the amount of lubricin in the control sample.

Examples of degenerative connecting tissue conditions that can be detected or diagnosed include, without limitation, tendonitis, osteoporosis, osteoarthritis, rheumatoid arthritis, gout, psoriatic arthritis, reactive arthritis, viral or post-viral arthritis, spondylarthritis, juvenile arthritis, systemic lupus erythematosus, camptodactyly-arthropathy-coxa vara-pericarditis syndrome (CACP), osteoporosis, or a connecting tissue condition caused by trauma.

In another aspect, the invention features a method for determining whether a substance modulates the level of lubricin in a connective tissue that includes (a) using the method of either the first or second aspect of the invention to measure the amount of lubricin in a test sample and in a control sample that contains lubricin, (b) contacting the test sample with a substance to be screened, (c) using the method employed in step (a) to detect lubricin in the test sample and in the control sample, (d) calculating the ratio of lubricin in the test sample to the amount of lubricin in the control sample for steps (a) and (c), and (e) determining that the substance modulates the level of lubricin in the connective tissue if the ratio of the amount of lubricin in the test sample to the amount of lubricin in the control sample is different in steps (a) and (c). In one embodiment, the sample includes lubricin-producing cells

In another aspect, the invention features a method of determining whether a subject would benefit from treatment for a degenerative joint condition that includes (a) obtaining a connecting tissue sample the subject, (b) using the method of either the first or second aspect of the invention to detect lubricin in the tissue sample and in a control sample, where the control sample contains a concentration of lubricin that is representative of that found in the tissue of a subject without a degenerative joint condition, (c) comparing the amount of lubricin in the tissue sample with the amount of lubricin in the control sample, (d) determining that a subject would benefit from treatment for a degenerative joint condition if the amount of lubricin in the subject's tissue sample is modulated compared to the amount of lubricin in the control sample.

In another aspect, the invention features a method for monitoring a subject's response to a treatment for a degenerative joint condition that includes (a) obtaining a first connecting tissue sample from the subject, (b) using the method of either the first or second aspect of the invention to detect lubricin in the first connecting tissue sample, (c) treating the subject for the condition, (d) obtaining a second connecting tissue sample from the subject, (e) using the same method used in step (b) to detect lubricin in the second sample, (f) comparing the amount of lubricin in the first tissue sample with the amount of lubricin in the second tissue sample, where a modulated amount of lubricin in the second sample compared to the first sample indicates that the subject has responded to the treatment.

In another aspect, the invention features a kit that includes (a) a first substance immobilized on a solid surface, where the first substance binds to a peptide sequence of lubricin; and (b) directions for using the solid surface in a sandwich-based binding assay. In one embodiment, the peptide sequence is substantially identical to one selected from the group consisting of: SEQ ID NO. 2, SEQ ID NO. 3, SEQ ID NO. 4, SEQ ID NO. 5, SEQ ID NO. 6, SEQ ID NO. 7, SEQ ID NO. 8, and SEQ ID NO. 9. Desirably, the peptide sequence is FESFERGRECDCDAQCKKYDK (SEQ ID NO. 2). In another embodiment, the kit further includes a second substance that binds to a carbohydrate moiety of lubricin.

In yet another aspect, the invention features a kit that includes (a) a first substance immobilized on a solid surface, where the first substance binds to a carbohydrate moiety of lubricin; and (b) directions for using the solid surface in a sandwich-based binding assay. In one embodiment, the first substance binds to a mucin-carbohydrate or to chondroitin. In another embodiment, the kit further includes a second substance that binds to a peptide sequence of lubricin, such as, for example, one selected from the consisting of: consisting of: SEQ ID NO. 2, SEQ ID NO. 3, SEQ ID NO. 4, SEQ ID NO. 5, SEQ ID NO. 6, SEQ ID NO. 7, SEQ ID NO. 8, and SEQ ID NO. 9, desirably SEQ ID NO. 2.

For any kit of the invention, the sandwich binding assay described by the directions can be either an ELISA or a surface plasmon resonance-based assay.

### DEFINITIONS

As used herein, the term “antibody” refers to a protein of the immunoglobulin (Ig) superfamily that binds noncovalently to certain substances (e.g., antigens and immunogens) to form an antibody-antigen complex. Antibodies can polyclonal (pAb), which can be obtained from an animal that is immunized with an antigen to elicit a polyclonal antibody response, or monoclonal (mAb), which can be obtained by recombinant methods, resulting in antibodies produced from hybridoma cells or other cell lines. It is understood that the term “antibody” as used herein includes within its scope any of the various classes or sub-classes of immunoglobulin derived from any of the animals or recombinant techniques that are obtained by conventional means (see, for example, Harlow and Jane, Antibodies, A Laboratory Manual, 1988, Cold Spring Harbor Publications, NY; Handbook of Monoclonal Antibodies, 1985, Ferone et al,. eds., Noges Publications, Park Ridge, N.J.; and Brodeur et al., Monoclonal Antibody Production Techniques and Applications, 1987, Marcel Dekker, Inc., NY).

As used herein, the term “aptamer” means a nucleic acid-based molecule that binds to another substance, where the binding is not determined by nucleic acid base pairing (see, for example, Jayasena, Clin. Chem. 45(9):1628-50, 1999 and Baldrich et al., Anal. Chem. 2004;76(23):7053-63).

As used herein, each of the terms “connecting tissue” or “connective tissue” means supportive tissue widely distributed in the body, characterized by large amounts of intercellular substance and relatively few cells. Non-limiting examples of “connecting tissue” or “connective tissue” include synovial fluid, synovium, tendon, tendon sheath, ligament, meniscus, intervertebral disc, chondrocytes, bone, and articular cartilage.

As used herein, the term “control sample” means either a sample obtained from a control subject, such as, for example, from the same subject before treatment or exposure to a foreign substance; from a second subject without a degenerative connecting tissue condition; or a sample that contains a known standard concentration of lubricin.

As used herein, the terms “degenerative connecting tissue condition,” “degenerative disease,” or “degenerative condition” includes a variety of connective tissue and joint diseases/disorders. Non-limiting examples include tendonitis, osteoporosis, arthritic conditions (e.g., osteoarthritis, rheumatoid arthritis, gout, psoriatic arthritis, reactive arthritis, viral or post-viral arthritis, spondylarthritis, juvenile arthritis, and systemic lupus erythematosus), CACP, osteoporosis, and or a connecting tissue conditions caused by trauma. Such degenerative joint diseases are characterized by morphological, compositional, and metabolic changes in articular cartilage. A subject with a degenerative joint disease may show clinical or subclinical signs of the disease, and thus demonstrate either early or late stages of the disease.

As used herein, the term “lubricin” means the glycoprotein having SEQ ID NO. 1 and containing at least 40% of the bound carbohydrate of the native protein. The term “lubricin” also includes those glycoproteins known as proteoglycan 4 (PRG4), articular cartilage superficial zone protein (SZP), megakaryocyte stimulating factor precursor, or tribonectin; in each case containing at least 40% of the bound carbohydrate of the native protein.

As used herein, each of the terms “modulated” or “modulation of” means either a significant increase or decrease in the level or amount of the indicated subject matter. Whether lubricin levels are increased or decreased in a subject with a degenerative condition depends on the particular sample and the status of the disease state. For example, with the onset of a degenerative joint disease, lubricin levels may transiently decrease in the synovial fluid, only to be followed by a compensatory increase. Such changes may occur when chondrocyte synthesis of lubricin decreases early in the disease process and is followed by a compensatory increase in synthesis and/or release by either chondrocytes or synovial cells. In another example involving the synovium or synovial fluid, the degenerative joint condition may be indicated by an elevated amount of deglycosylated lubricin in the test sample but indicated by a decreased level of intact lubricin in articular cartilage or on the articular surface.

As used herein, the term “osteoarthritis,” includes both primary and secondary degenerative joint disease. A subject with osteoarthritis may show any of the early manifestations of osteoarthritis, including, for example, increased water content of the cartilage, increased collagen extractability, increased levels of annexin V, crepitus, and radiologic changes (including joint space narrowing, subchondral sclerosis or cysts, and osteophyte formation), or later manifestations, including, for example, joint pain, joint swelling, joint stiffness, reduced quality and quantity of cartilage matrix, deformity, chondrocalcinosis, and reduced range of motion.

As used herein, the term “rheumatoid arthritis” refers to inflammatory joint disease in both early and late stages. Signs and manifestations of the early stages include, for example, general fatigue, joint stiffness or aching, synovial inflammation, excessive synovial fluid, joint effusion, osteoporosis in the ends of the bones forming the affected joint or joints, edematous synovial cells, and proliferation of synovial lining cells. In later stages, additional signs and manifestations are detected, including joint pain, redness, swelling, and inflammation. Pannus can be seen in the joints. Cartilage and subchondral bone can be eroded at the articular surface. Changes in the composition of the synovial fluid can occur. Laxity in tendons and ligaments, as well as deformity, can occur and can cause limitations in joint range of motion and joint instability. Furthermore, Rheumatoid Factor(s) can be detected in the subject's blood at both early and late stages of the disease.

As used herein, the terms “sandwich-based binding assay” or “sandwich assay” mean a qualitative or quantitative assay that features two separate binding elements for the substance being assayed. For example, the present invention features a sandwich assay that includes two binding elements for lubricin, one of which binding to a peptide sequence in lubricin and the other of which binding to a carbohydrate moiety of lubricin.

As used herein, the term “subject” means an animal. Preferably the subject is a mammal, and more preferably, a human. Subject animals also include domesticated animals such as, for example, cats and dogs; livestock, such as, for example, cattle, horses, pigs, sheep, and goats; and laboratory animals, such as, for example, mice, rabbits, rats, guinea pigs, primates, and dogs.

As used herein, the term “substance that binds to a peptide sequence” means a substance that binds to one or more amino acids of a defined peptide sequence, and includes those substances that bind to non-contiguous as well as contiguous peptide sequences. For example, a substance that binds to a non-contiguous peptide sequence of lubricin binds to one or more amino acids of a defined peptide sequence and also to one or more amino acids in another undefined peptide sequence, where the undefined peptide sequence is close to the defined peptide sequence in the three-dimensional structure of the protein.

As used herein, “substantially identical” refers to a peptide or nucleic acid sequence exhibiting at least 75%, but preferably 85%, more preferably 90%, most preferably 95%, or even 99% identity to a reference peptide or nucleic acid sequence. For peptides, the length of comparison sequences will generally be at least 20 amino acids. For nucleic acids, the length of comparison sequences will generally be at least 60 nucleotides, preferably at least 90 nucleotides, and more preferably at least 120 nucleotides.

### BRIEF DESCRIPTION OF THE DRAWING

FIG. 1 is a diagram showing the concentration of lubricin in the synovial fluid of rabbits, as assayed at 1, 2, and 3 week time periods in an injured rabbit model.

### DETAILED DESCRIPTION

The present invention provides sandwich assays for the detection of lubricin in a sample. The invention also features the use of such an assay for diagnosing degenerative connecting tissue conditions, determining whether a substance modulates the level of lubricin in a connective tissue, determining whether a subject benefits from a treatment for a degenerative joint condition, and for monitoring a subject's response to a treatment for a degenerative joint condition. Also featured are kits used to perform an assay of the invention.

Sandwich Assays/ELISA

A sandwich assay of the invention may be conducted according to any of the known sandwich assay formats. In one example of a sandwich assay of the invention, a first substance that binds to a peptide sequence on lubricin (the capture substance) is immobilized on a solid surface. The surface is then contacted with a sample (or sample aliquot) that is suspected of containing lubricin, under conditions whereby a complex between the first substance and lubricin can form. The surface is then contacted to a second substance (the detection substance) which binds a carbohydrate moiety of lubricin, such as, for example, a mucin carbohydrate. Alternatively, the first substance can be one that binds a carbohydrate moiety on lubricin and the second substance one that binds to a lubricin peptide sequence.

In addition to being able to bind a lubricin carbohydrate, the second substance may be linked to a reporter group, the signal of which is used to indicate the binding of the second substance to any lubricin bound to the solid surface. By “reporter group”, as used herein, is meant any group that provides an analytically detectable signal that allows for the detection of lubricin. Preferably, detection is quantifiable, such that the amount of lubricin in the sample may be calculated in absolute terms, or may be done in comparison with a standard (or series of standards) containing a known amount of lubricin. Assays that involve the use of capture substances and detection substances that include a reporter group include, without limitation, sandwich ELISAs, sandwich Western blotting assays, and sandwich immunomagnetic detection assays.

Methods of detecting or quantitating a reporter group with a detectable label depend on the nature of the label and are known in the art. Appropriate labels include, without limitation, radionuclides (e.g., ¹²⁵I, ¹³¹I, ³⁵S, , ³H, ³²P, ³³P, or ¹⁴C); fluorescent moieties (e.g., fluorescein, rhodamine, or phycoerythrin); luminescent moieties (e.g., Qdot™ nanoparticles supplied by the Quantum Dot Corporation, Palo Alto, Calif.); or enzymes (e.g., alkaline phosphatase or horseradish peroxidase). The products of reactions catalyzed by appropriate enzymes can be, without limitation, fluorescent, luminescent, or radioactive, or they may absorb visible or ultraviolet light. Examples of detectors include, without limitation, x-ray film, radioactivity counters, scintillation counters, spectrophotometers, colorimeters, fluorometers, luminometers, and densitometers.

The most commonly used reporter groups in sandwich assays are either enzymes or fluorophores. When enzymes are used, they can conjugated to the detection substance, often by means of glutaraldehyde or periodate. As will be readily recognized, however, a wide variety of different conjugation techniques exist, which are well known to the skilled artisan. Commonly used enzymes include horseradish peroxidase, glucose oxidase, β-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. In all cases, the enzyme-labeled detection substance is added to the capture substance-lubricin complex and allowed to bind to the complex, and then the excess reagent is washed away. A solution containing the appropriate substrate is then added to the tertiary complex of detection substance-antigen-capture substance. The substrate reacts with the enzyme linked to the detection substance, giving a signal, which may be further quantitated, usually spectrophotometrically, to give an evaluation of the amount of antigen that is present in the sample.

Alternately, fluorescent compounds, such as fluorescein or rhodamine, may be chemically coupled to the detection substance without altering its binding capacity. When activated by illumination with light of a particular wavelength, the fluorochrome-label absorbs the light energy, inducing a state of excitability in the molecule, followed by emission of the light at a characteristic longer wavelength. The emission appears as a characteristic detectable color. As in the enzyme assay described above, the fluorescent-labeled detection substance is allowed to bind to the capture substance-lubricin complex. After washing the unbound reagent, the remaining ternary complex is then exposed to light of the appropriate wavelength, with the fluorescence observed indicating the presence of the antigen.

Suitable solid surfaces to which the capture substance can be bound include any suitable support for a binding reaction and/or any surface to which molecules may be attached through either covalent or non-covalent bonds. Examples, without limitation, include glass plates; functionalized glass; germanium; silicon; gallium arsenide; gold; silver; palladium; copper; aluminum; PTFE, membranes, such as nylon or nitrocellulose membranes; and polymeric substances (e.g., without limitation, agarose, cellulose, polystyrene, polyvinylchloride, polypropylene, or polyacrylamide). Any other material known in the art that is capable of having functional groups such as amino, carboxyl, thiol or hydroxyl incorporated on its surface, is also contemplated. This includes surfaces with any topology, including, but not limited to, flat surfaces, spherical surfaces, grooved surfaces, particles, and cylindrical surfaces, such as, for example, columns.

Surface Plasmon Resonance Sandwich Assay

Alternatively, the second substance is not linked to a reporter group and its binding to lubricin is assessed by another means, such as, for example, a surface plasmon resonance measurement.

Surface plasmon resonance (SPR) is the phenomenon that is caused by the resonance of a surface plasmon wave, which is induced along the metal surface by light incident on a metal layer, and an evanescent wave, which is generated by the incident light (see Mullett et al., Surface plasmon resonance-based immunoassays, Methods 22(1):77-91, 2000; Rich and Myszka, Advances in surface plasmon resonance biosensor analysis, Current Opinion in Biotechnology 11(1):54-61, 2000; and U.S. Pat. Nos. 4,844,613; 4,889,427; 5,341,215; and 6,831,748). The surface plasmon resonance is dependent on the wavelength and the angle of the incident light. Characteristically, when surface plasmon resonance is excited, the optical component having a specific incident angle or a specific wavelength transfers its optical energy to the surface plasmon wave, and the reflected optical component that has the corresponding incident angle or wavelength therefore decreases markedly.

In order to induce the surface plasmon resonance, it is necessary to use a metal, such as, for example, gold, silver, copper, aluminum, or palladium, to generate a specific surface plasmon wave and an optical structure for inducing an evanescent wave that can be resonant with the surface plasmon wave. As the examples of the optical structure for inducing an evanescent wave, two kinds of structures are currently known: an optical structure utilizing total reflection with a prism, and an optical structure utilizing a diffraction grating. When a metal component is combined with one of these optical structures it is commonly called a surface plasmon resonance sensor chip.

In an example of the present invention that takes advantage of SPR, a first substance (the capture substance) that binds to a peptide sequence of lubricin is immobilized on a metal film on the surface of a prism, followed by contacting the film with a sample containing, or suspected of containing lubricin. The metal film is then incubated in an appropriate liquid medium such that the metal film is at the prism-liquid interface. Light is directed through the prism towards the medium, and at a critical angle, total internal reflection of the light occurs. Above this critical angle, an evanescent wave extends into the medium by a distance that is approximately equal to the wavelength of the incident light. The evanescent wave excites free oscillating electrons, termed surface plasmons, in the metal film, and causes them to resonate. Energy is absorbed from the evanescent wave by the electrons during this process, thereby reducing the intensity of the internally reflected light. The angle at which total internal reflection, and hence resonance, occurs is exquisitively sensitive to changes in the refractive index of the medium immediately adjacent to the metal film. When a second substance binds to the complex formed between lubricin and the capture substance on the surface of the film, the refractive index at this site changes, and the angle needed to cause resonance changes also. In the present invention, as example of a second substance is one that binds to a lubricin carbohydrate moiety. In order to detect the binding of the second substance, a detector system is arranged in which the angle of incident light is varied, and the intensity of the reflected light is measured. Resonance occurs when the intensity of the reflected light is at a minimum. Any perturbation observed in the angle at which this minimum occurs is related to the surface concentration of the second substance. An example of a device for measuring SPR, the BIAcore™ sensor chip, is commercially available from Pharmacia Biosensors.

In another example of the invention in which SPR can be used to assess the concentration of lubricin in a sample, the capture substance is one that binds to a carbohydrate moiety of lubricin and the second substance binds to a peptide sequence of lubricin.

In addition to, or as an alternative to, the use of metal films for the support of the lubricin capture substance in an SPR assay of the invention, the second lubricin-binding substance can include a colloidal metal particle, such as, for example, colloidal gold or silver, coated with the binding moiety (see, for example, Cooper, J. Mol. Recognit. 17(4):286-315, 2004; Haes et al., J. Am. Chem. Soc. 127(7):2264-71, 2005; Liu et al., Anal. Biochem. 333(1):99-104, 2004; Pieper-Furst et al., Anal. Biochem. 332(1):160-7, 2004; Schultz et al., Proc. Natl. Acad. Sci. USA. 97(3):996-1001, 2000; and U.S. Pat. Nos. 4,979,821 and 6,180,415). As before, the capture substance can either bind to a lubricin peptide sequence or a lubricin carbohydrate moiety, with the second binding substance of the sandwich assay reacting with a lubricin carbohydrate moiety or a lubricin peptide sequence, respectively.

Kits

The antibodies and revealing reagents for the conduct of an assay of the invention using standard detection protocols, for example radioisotope labeling, fluorescent labeling, ELISA, or a surface plasmon resonance measurement may conveniently be supplied as kits that include the necessary components and instructions for the assay. In one embodiment of the invention such a kit can include a microtitre plate coated with a first substance (e.g., an antibody) that binds to a peptide sequence selected from the group consisting of: SEQ ID NO. 2, SEQ ID NO. 3, SEQ ID NO. 4, SEQ ID NO. 5, SEQ ID NO. 6, SEQ ID NO. 7, SEQ ID NO. 8, and SEQ ID NO. 9; standard solutions for preparation of standard curve; a second substance (e.g., peanut agglutinin) reactive with a lubricin carbohydrate and conjugated to an enzyme such as horse radish peroxidase; a substrate solution; a stopping solution; a washing buffer; and instructions for performing the assay.

### EXAMPLES

### Example 1

### Generation of Polyclonal Antibody to SEQ ID NO. 2

FESFERGRECDCDAQCKKYDK is prepared by standard peptide synthesis methodology (see Bodansky and Trost, Ed., Principles of Peptide Synthesis, 1993, Springer-Verlag Inc., NY; Grant, Synthetic Peptides: A User Guide, 1992, W.H. Freeman and Co., NY)

An emulsion of PBS (500 μL) containing a KLH conjugate of FESFERGRECDCDAQCKKYDK (1 mg sample) and FCA (500 μL) IS subcutaneously administered to a male New Zealand White Rabbit aged seven weeks. After about 4 weeks, the rabbit is sensitized four additional times for the following 2 weeks by this PBS emulsion along with FICA. After the first, third and fifth immunization by the antigen for immunization and FICA, the titer of antibody in blood is measured as follows.

Blood is collected from the rabbit and a serum sample is prepared. To 50 μL of the serum sample is added successively water (450 μL), 0.5% γ-globulin (80 μL) and a saturated aqueous solution of ammonium sulfate (580 μL), and then the mixture is centrifuged. The pellet is dissolved into 50 mM PB (200 μL), and 50 mM PB (100 μL) containing ¹⁴C—FESFERGRECDCDAQCKKYDK (400 Bq) is added, followed by incubation at 4° C. overnight. To the mixture is added successively 0.1M PB (200 μL) containing 0.5% bovine .gamma.-globulin and 30% polyethylene glycol 6000 (register trademark, Wako Pure Chem. Ind. Ltd., 600 μL), and then the mixture is centrifuged. Radioactivity of the pellet is measured. After the third immunization, a sufficiently high titer of antibody in blood serum, referred to as pAB J108, is obtained for further use.

### Example 2

### Sandwich ELISA Assay for Lubricin in Rabbit Synovial Fluid

Microtiter plates (Corning, Corning, N.Y.) were coated with pAB J108 and left standing overnight at 4° C. pAB J108 is a rabbit polyclonal antibody generated against FESFERGRECDCDAQCKKYDK, a polypeptide representing the SEQ ID NO. 1 epitope, which is encoded by PRG 4 exon 3, which encodes the amino terminus present in all lubricin isoforms. The solution used to coat the plate was a 1:10,000 dilution of antibody in 10 mM sodium carbonate buffer, pH=9.0. Each well received 100 μL. The plate was subsequently washed twice (250 μL/each well) with phosphate buffered saline (PBS)+0.05% Tween20®. The plate was then incubated with bovine serum albumin (BSA, Sigma-Aldrich, St. Louis, Mo.) in carbonate buffer (pH=9.0, 200 μL/well) for 2 hours at room temperature.

Human lubricin, obtained from patients undergoing total knee replacement and purified by a previously described method (Jay et al., J. Orthop. Res 2001;19:677-87), was used to construct the standard curve. Serial dilutions of human lubricin (5.0 to 0.025 μg/mL), normal rabbit synovial fluid (SF), and SF from injured rabbit knees (100 μL/well) were individually placed in wells of the microtiter plate and incubated at room temperature for 1 hour. The plate was washed twice with PBS+0.05% Tween20® (250 μL/well), followed by treatment with peanut agglutinin (PNA; from Arachis hypogaea) conjugated to peroxidase (Sigma-Aldrich) at a concentration of 0.25 μg/mL in PBS/1% BSA (100 μL/well) for 1 hour at room temperature. The plate was washed twice with PBS+0.05% Tween20® (250 μL/well) and twice with PBS (100 μL/well). Fluorescence substrate developing solution (100 μL, Quanta Blue; Pierce, Rockford, Ill.) was added for 1 hour, and the fluorescence was measured at an excitation wavelength of 330 nm and an emission wavelength of 460 nm using a Packard fluorocounter (Packard, Meriden, Conn.).

### Example 3

### Concentration of Lubricin in a Rabbit Injury Model

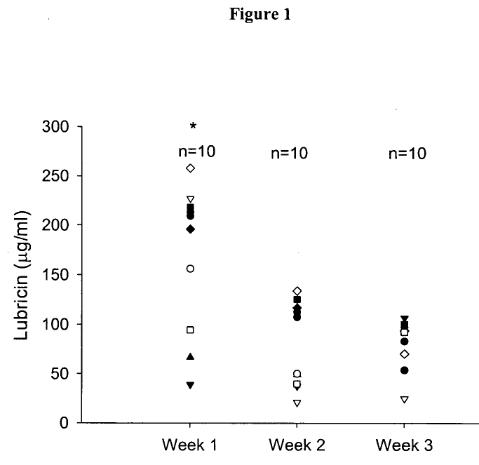

The rabbit injury model used was adapted (see Rogart et al., Osteoarthritis Cartilage 7:539-547, 1999) from the that described by Hulth and Telhag (see Hulth et al., Acta Orthop. Scand. 41:552-530, 1970). A total of 10 New Zealand white rabbits were used. The hind right knee of each rabbit underwent anterior cruciate ligament and posterior cruciate ligament transections. The hind left knee received a sham operation. Each rabbit underwent operative knee joint aspirations, under aseptic conditions, at one week intervals for three weeks. The presence of post-traumatic effusions obviated the need for lavage with saline. Approximately 600 μL of SF was collected from each rabbit after each weekly interval. The SF was centrifuged, separated, and stored immediately at −20° C. Lavage fluid was also aspirated, after each one week interval for three weeks, from the sham knee. A total of 1.0 mL of sterile saline was injected into the sham knee joint. The joint was mobilized through ten full-arc flexion/extension ranges of motion and lavage fluid, 1.0 mL was subsequently aspirated and stored at −20° C. for a baseline articular damage markers analysis. Additional normal rabbit SF (30 μL) was obtained post-mortem by aspirating the knees of 8 New Zealand white rabbits. The results, shown in FIG. 1, indicate that lubricin concentrations in synovial fluid were significantly higher at week 1 than at weeks 2 and 3.

All publications and patents cited in this specification are hereby incorporated by reference herein as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for detecting lubricin in a sample, said method comprising the steps of:
(a) immobilizing on a solid surface a first substance that binds to a peptide sequence of lubricin;
(b) contacting said solid surface having said first substance immobilized thereto with a sample suspected of containing lubricin, under conditions in which lubricin binds to said first substance;
(c) contacting said solid surface with a second substance that binds to a carbohydrate moiety of lubricin, under conditions in which said carbohydrate binds to said second substance; and
(d) detecting lubricin in said sample by observing the binding of said second substance to said solid surface.

2. The method of claim 1, wherein said sample comprises synovial fluid, synovium, tendon, tendon sheath, ligament, meniscus, intervertebral disc, chondrocytes, or articular cartilage.

3. The method of claim 1, wherein said peptide sequence is substantially identical to a peptide sequence selected from the group consisting of: SEQ ID NO. 2, SEQ ID NO. 3, SEQ ID NO. 4, SEQ ID NO. 5, SEQ ID NO. 6, SEQ ID NO. 7, SEQ ID NO. 8, and SEQ ID NO. 9.

4. The method of claim 3, wherein said peptide sequence is SEQ ID NO. 2.

5. The method of claim 1, wherein said first substance comprises an antibody that binds to said peptide sequence.

6. The method of claim 5, wherein said antibody is polyclonal.

7. The method of claim 5, wherein said antibody is monoclonal.

8. The method of claim 1, wherein said first substance comprises a nucleic acid that binds to said peptide fragment.

9. The method of claim 8, wherein said nucleic acid is an RNA-based aptamer.

10. The method of claim 1, wherein said second substance binds a mucin carbohydrate.

11. The method of claim 10, wherein said second substance comprises peanut agglutinin.

12. The method of claim 10, wherein said second substance comprises an antibody.

13. The method of claim 1, wherein said second substance binds chondroitin.

14. The method of claim 13, wherein said second substance comprises an antibody.

15. The method of claim 1, wherein said second substance is a secondary reagent in an ELISA.

16. The method of claim 15, wherein said second substance comprises peroxidase.

17. The method of claim 1, wherein said solid surface is a sensing surface for surface plasmon resonance-based measurements.

18. The method of claim 1, wherein said second substrate is attached to a plasmon resonance particle.

19. A method of detecting or diagnosing a degenerative connecting tissue condition in a subject, said method comprising the steps of:
(a) obtaining a connecting tissue sample from said subject;
(b) detecting lubricin in said tissue sample and in a control sample by the method of claim 1;
(c) comparing the amount of lubricin in said tissue sample with the amount of lubricin in said control sample; and
(d) determining that said subject has a degenerative connecting tissue condition if the amount of lubricin in said tissue sample is modulated compared to the amount of lubricin in said control sample.

20. The method of claim 19, wherein said degenerative connecting tissue condition is tendonitis, osteoporosis, osteoarthritis, rheumatoid arthritis, gout, psoriatic arthritis, reactive arthritis, viral or post-viral arthritis, spondylarthritis, juvenile arthritis, systemic lupus erythematosus, camptodactyly-arthropathy-coxa vara-pericarditis syndrome (CACP), osteoporosis, or a connecting tissue condition caused by trauma.

21. The method of claim 19, wherein the method of claim 1 comprises an ELISA measurement to determine the amount of said second substance bound to said solid surface.

22. The method of claim 19, wherein the method of claim 1 comprises a surface plasmon resonance measurement to determine the amount of said second substance bound to said solid surface.

23. A method of determining whether a substance modulates the level of lubricin in a connective tissue, said method comprising:
(a) contacting a test sample with said substance, wherein said test sample contains lubricin;
(b) detecting lubricin in said test sample, and in a control sample that has not been contacted to said substance, by the method of claim 1;
(c) comparing the amount of lubricin in said test sample with the amount of lubricin in said control sample; and
(d) determining that said substance modulates the level of lubricin if the amount of lubricin in said test sample is different that the amount of lubricin in said control sample.

24. The method of claim 23, wherein said test sample in step (a) further comprises lubricin-producing cells.

25. The method of claim 23, wherein the method of claim 1 comprises an ELISA measurement.

26. The method of claim 23, wherein the method of claim 1 comprises a surface plasmon resonance measurement.

27. A method of determining if a subject would benefit from treatment for a degenerative joint condition, said method comprising:
(a) obtaining a connecting tissue sample from said subject;
(b) detecting lubricin in said tissue sample and in a control sample by the method of claim 1;
(c) comparing the amount of lubricin in said tissue sample with the amount of lubricin in said control sample; and
(d) determining that said subject would benefit from treatment for a degenerative joint condition if the amount of lubricin in said tissue sample is modulated compared to the amount of lubricin in said control sample.

28. The method of claim 27, wherein the method of claim 1 comprises an ELISA measurement.

29. The method of claim 27, wherein the method of claim 1 comprises a surface plasmon resonance measurement.

30. A method of monitoring a subject's response to a treatment for a degenerative joint condition, said method comprising:
(a) obtaining a first connecting tissue sample from said subject;
(b) detecting lubricin in said first connecting tissue sample by the method of claim 1;
(c) treating said subject for said condition;
(d) obtaining a second connecting tissue sample from said subject;
(e) detecting lubricin in said second connecting tissue sample by the method of claim 1; and
(f) comparing the amount of lubricin in said first connecting tissue sample with the amount of lubricin in said second connecting tissue sample, wherein a modulated amount of lubricin in said second sample compared to said first sample indicates a response to said treatment.

31. The method of claim 30, wherein the method of claim 1 comprises an ELISA measurement to determine the amount of said second substance bound to said solid surface.

32. The method of claim 30, wherein the method of claim 1 comprises a surface plasmon resonance measurement to determine the amount of said second substance bound to said solid surface.

33. A kit comprising (a) a first substance immobilized on a solid surface, wherein said first substance binds to a peptide sequence of lubricin; and (b) directions for using said solid surface in a sandwich-based binding assay.

34. The kit of claim 33, wherein said peptide sequence is substantially identical to one selected from the group consisting of: SEQ ID NO. 2, SEQ ID NO. 3, SEQ ID NO. 4, SEQ ID NO. 5, SEQ ID NO. 6, SEQ ID NO. 7, SEQ ID NO. 8, and SEQ ID NO. 9.

35. The kit of claim 33, wherein said peptide sequence is SEQ ID NO. 2.

36. The kit of claim 33, wherein said kit further comprises a second substance that binds to a carbohydrate moiety of lubricin.

37. The kit of claim 33, wherein said sandwich-based binding assay is an ELISA assay.

38. The kit of claim 33, wherein said sandwich-based binding assay is a surface plasmon resonance assay.

39. The method claim 33, wherein said second substrate is attached to a plasmon resonance particle.


SEQUENCE LISTING (9 sequences)

SEQ ID NO: 1 - protein, 1404 aa, Homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIWKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTAPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTSDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGEMTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 2 - protein, 21 aa, Homo sapiens
FESFERGRECDCDAQCKKYDK

SEQ ID NO: 3 - protein, 25 aa, Homo sapiens
MAWKTLPIYLLLLLSVFVIQQVSSQ

SEQ ID NO: 4 - protein, 25 aa, Homo sapiens
LLLLSVFVIQQVSSQDLSSCAGRCG

SEQ ID NO: 5 - protein, 25 aa, Homo sapiens
QVSSQDLSSCAGRCGEGYSRDATCN

SEQ ID NO: 6 - protein, 25 aa, Homo sapiens
AGRCGEGYSRDATCNCDYNCQHYME

SEQ ID NO: 7 - protein, 25 aa, Homo sapiens
DATCNCDYNCQHYMECCPDFKRVCT

SEQ ID NO: 8 - protein, 25 aa, Homo sapiens
QHYMECCPDFKRVCTAELSCKGRCF

SEQ ID NO: 9 - protein, 25 aa, Homo sapiens
KRVCTAELSCKGRCFESFERGRECD